(19) United States
(12) Patent Application Publication
(10) Pub. No.: US 2025/0090622 A1
(43) Pub. Date: Mar. 20, 2025

(54) ANGIOTENSIN COMPOSITIONS AND METHODS RELATED THERETO

(71) Applicant: LA JOLLA PHARMA, LLC, Waltham, MA (US)

(72) Inventor: James Rolke, Tucson, AZ (US)

(21) Appl. No.: 18/813,897

(22) Filed: Aug. 23, 2024

Related U.S. Application Data
(63) Continuation of application No. 17/592,943, filed on Feb. 4, 2022.
Application No. 17/592,943 is a division of application No. 16/220,901, filed on Dec. 14, 2018.
(60) Provisional application No. 62/599,606, filed on Dec. 15, 2017.

(51) Int. Cl. A61K 38/08; A61K 9/00; A61P 9/02; C07K 7/14; C07K 14/575

(52) U.S. Cl. CPC A61K 38/085; A61K 9/0019; A61P 9/02; C07K 7/14; C07K 14/575

(57) ABSTRACT

Provided herein are dosage forms and kits comprising angiotensin II that are suitable for the treatment of low blood pressure. In particular, dosage forms and kits that facilitate the ability to rapidly prepare angiotensin II for IV infusion into a subject.

### RELATED APPLICATIONS

This application is a continuation of U.S. patent application Ser. No. 17/592,943, filed

Feb. 4, 2022, which is a divisional of U.S. patent application Ser. No. 16/220,901, filed Dec. 14, 2018, which claims the benefit of priority to U.S. Provisional Patent Application Ser. No. 62/599,606, filed Dec. 15, 2017, the contents of each of said applications are herein incorporated by reference in its entirety.

### BACKGROUND

A basic treatment goal in patients with circulatory shock, such as those with distributive or vasodilatory shock, is the rapid restoration and maintenance of effective perfusion to vital organs and tissues prior to the onset of cellular injury. Effective tissue perfusion depends on both sufficient cardiac output and adequate driving pressure, thus treatment of circulatory shock requires maintenance of an appropriate cardiac index and mean blood pressure. Circulatory shock is associated with a significant mortality rate, and the mortality rate is even higher among patients whose blood pressure remains low despite treatment with fluids and catecholamines.

Angiotensin II, which is a naturally-occurring octa-peptide hormone that plays a central role in cardiovascular homeostasis, is a vasoactive agonist that induces constriction of blood vessels. Angiotensin II can constrict both arteries and veins and can increase blood pressure. Intravenous dosing of angiotensin II in rats, dogs, and rabbits has been shown to produce an increase in blood pressure. Correa et al. (Critical Care Medicine. 42 (8): e550-9, (2014)) compared the effects of norepinephrine (NE) and angiotensin II on hemodynamics, organ function, and mitochondrial respiration in porcine fecal peritonitis septic shock model and showed that angiotensin II reversed sepsis-induced hypotension with systemic and regional hemodynamic effects similar to those of NE. Angiotensin II is being evaluated for treatment of hypotension in humans.

Therefore, improved pharmaceutical compositions containing angiotensin II are desired. In particular, pharmaceutical compositions that facilitate the ability to rapidly prepare angiotensin II for infusion are desirable.

### SUMMARY

Distributive shock (also referred to as vasodilatory shock) is a medical condition in which low blood pressure results in inadequate supply of blood to the body's tissues and organs. Common causes of distributive shock are sepsis, inflammation, vasoplegia, and/or severe drug reactions. Physicians typically maintain patients with shock at a mean arterial pressure (MAP) of at least 65 mmHg using fluid replacement and, if that is insufficient, then vasopressor treatment. Commonly used vasopressors include catecholamines and vasopressins; however, a significant percentage of patients fail to respond to these treatments. Even short durations of hypotension (e.g., 1-5 minutes) are associated with increased severe adverse events such as myocardial infarction, stroke, and acute kidney injury.

In addition, prolonged exposure to elevated plasma levels of catecholamines may result in significant adverse effects. Adrenergic overstimulation induces cardiac toxicity, immunosuppression, decreased renal blood flow, and severe peripheral ischemia.

Angiotensin II is a peptide hormone naturally produced by the body that regulates blood pressure through a third system, the renin-angiotensin system (RAS), via vasoconstriction and sodium reabsorption. Angiotensin II is a potent, direct vasoconstrictor that increases MAP, but its serum half-life is extremely short, necessitating continuous delivery during treatment, e.g., by a continuous intravenous infusion.

Currently, there is only a single commercial supplier of angiotensin suitable for parenteral administration, selling vials of 0.050 mg angiotensin powder. This vial size reflects the potency of angiotensin II and historical usage patterns. However, it has been found that treatment of distributive shock in a single patient requires an average daily dose of angiotensin II of about 4 mg/day. Preparing an IV solution to treat a patient for a day would require dissolving the contents of dozens of vials of the commercial angiotensin II, a time-consuming effort that introduces potential contamination from all of the handling, and dosing errors if the contents of each vial are not completely dissolved and transferred, or if the amount of angiotensin II in each vial is not precisely known.

Since time to administration of treatment is crucial for patient survival, angiotensin II must be packaged for rapid preparation of the drug for administration in an efficient and reliable way.

Accordingly, provided herein are formulations and compositions of angiotensin II that are suitable for the rapid treatment of low blood pressure (for example, vasodilatory shock). In certain embodiments, formulations disclosed herein reduce the time to begin treatment. In certain embodiments, such formulations and compositions are packaged such that each unit contains an amount of angiotensin II that facilitates its usage as a treatment for hypotension. In certain embodiments, angiotensin II is provided as a lyophilisate (e.g., a freeze-dried powder) in a vial comprising about 0.5 mg/vial to about 20 mg/vial. The vial size may range from 1 mL to 100 mL to allow for dissolution to a variety of concentrations ranging from 0.005 mg/mL to 20 mg/mL. Preferred vial size for the compositions disclosed herein include 1 mL vials and 2 mL vials.

In preferred embodiments, angiotensin II is provided in a liquid formulation in a vial comprising about 0.5-20 mg/mL angiotensin II. Preferably, the vial has a volume between 0.5 and 100 mL, more preferably between 1 and 20 mL, and most preferably between 1 and 5 mL, such as 1 mL or 2 mL.

In another aspect, angiotensin II is supplied as or can be diluted to a liquid formulation in an IV infusion bag or bottle at a concentration of about 0.00005 mg/mL to about 0.01 mg/mL, e.g., to allow for continuous, uninterrupted infusion for at least 24 hours. In other embodiments, continuous, uninterrupted infusion lasts for at least 6 to 48 hours. In certain embodiments, continuous, uninterrupted infusion can last for at least 12 to 24 hours. In other preferred embodiments continuous, uninterrupted infusion lasts for at least 24 to 48 hours. In yet other embodiments, continuous, uninterrupted infusion lasts for at least 48 hours.

In certain embodiments, the formulations of the invention have a pH from about 2 to about 8, preferably, from about 3 to about 6, more preferably, from about 4 to about 6, and even more preferably, from about 5 to about 6. In some preferred embodiments, the pH is between 5 to 6, particularly, about 5.5.

In further aspects, provided herein are kits, packages and multi-container units containing the herein described pharmaceutical compositions and/or means for administering the same for use in the treatment of subjects. Briefly, these kits include a container or formulation that contains angiotensin II, or pharmaceutically acceptable salts thereof.

### DETAILED DESCRIPTION

### General

Angiotensin II is a promising therapeutic to increase blood pressure, e.g., to treat hypotension, such as catecholamine-resistant hypotension, septic shock, vasodilatory shock and high-output shock. Angiotensin II has a half-life in circulation of approximately 30 seconds and a half-life as long as 15 to 30 minutes in tissues. Angiotensin II increases secretion of antidiuretic hormone (ADH) and adrenocorticotropin hormone (ACTH), and may potentiate sympathetic effects by direct action on postganglionic sympathetic fibers. Angiotensin II also acts on the adrenal cortex, causing it to release aldosterone.

In certain aspects, provided herein are formulations of angiotensin II, and kits thereof, that facilitate its rapid and/or ready use as a treatment for hypotension. Angiotensin II formulations described herein can be administered to a subject in need of a treatment of low blood pressure.

### Definitions

For convenience, certain terms employed in the specification, examples, and appended claims are collected here.

As used herein, the singular forms “a,” “an” and “the” are intended to include the plural forms as well, unless the context clearly indicates otherwise.

The term “about” means within an acceptable error range for the particular value as determined by one of ordinary skill in the art, which will depend in part on how the value is measured or determined, i.e., the limitations of the measurement system. Where the terms “about” or “approximately” are used in the context of compositions containing amounts of ingredients or conditions such as temperature, these values include the stated value with a variation of 0-10% around the value (X±10%).

The terms “including,” “includes,” “having,” “has,” “with,” or variants thereof are inclusive in a manner similar to the term “comprising.” The term “consisting” and the grammatical variations of “consist” encompass embodiments with only the listed elements and excluding any other elements. The phrases “consisting essentially of” or “consists essentially of” encompass embodiments containing the specified materials or steps and those including materials and steps that do not materially affect the basic and novel characteristic(s) of the embodiments.

Ranges are stated in shorthand to avoid having to set out at length and describe each and every value within the range. Therefore, when ranges are stated for a value, any appropriate value within the range can be selected, and these values include the upper value and the lower value of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc.

For the purposes of this invention the term “pharmaceutically effective amount” of angiotensin II refers to the amount of the angiotensin II which, when administered to a subject, elicits a therapeutic response in the subject including an increase in blood pressure that reduces or eradicates one or more of the physiological symptoms associated with hypotension, notwithstanding that the patient may still be afflicted with the underlying disorder.

The term “hypotension” refers to low blood pressure in a subject, for example, a human, particularly, in the arteries of the systemic circulation. Typically, a human suffering from hypotension exhibits a systolic blood pressure of less than 90 millimeters of mercury (mm of Hg) and/or diastolic blood pressure of less than 60 mm of Hg. Because of varied physiology among different individuals, a human having a systolic blood pressure of less than 90 mm of Hg and/or diastolic blood of less than 60 mm of Hg may not always exhibit symptoms of hypotension. Therefore, hypotension in a subject also indicates a blood pressure that causes noticeable symptoms of low blood pressure in a subject; which may include chest pain, shortness of breath, irregular heartbeat, loss of consciousness, profound fatigue, or temporary blurring or loss of vision.

“Pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with angiotensin II, its use in the pharmaceutical formulations of the invention is contemplated. In certain embodiments, the pharmaceutically acceptable excipient is a saline solution.

“Treatment” and grammatical variants of treatment refer to an approach for obtaining beneficial or desired results including but not limited to therapeutic benefit. A therapeutic benefit is achieved with the eradication, reduction, or amelioration of one or more of the physiological symptoms associated with the underlying disorder such that an improvement is observed in the patient, notwithstanding that the patient may still be afflicted with the underlying disorder.

“Subject” refers to an animal, such as a mammal, for example a human. The methods described herein can be useful in both humans and non-human animals. In some embodiments, the subject is a mammal (such as an animal model of disease), and in some embodiments, the subject is human. The subject can also be a rodent, lagomorph, feline, canine, porcine, ovine, bovine, equine, or primate. The subject may be a female or male. The subject may be an infant, child, or adult.

### Dosage Forms of Angiotensin II

Angiotensin II used in the formulations described herein can be a human angiotensin II or a mammalian angiotensin II, for example, angiotensin II from a rodent, a feline, a canine, a porcine, an ovine, a bovine, an equine, or a primate.

In certain embodiments, angiotensin II is a human angiotensin II, which is an octa-peptide having the amino acid sequence of H-Asp-Arg-Val-Try-Ile-His-Pro-Phe-OH (SEQ ID NO: 1), and has the following chemical structure:

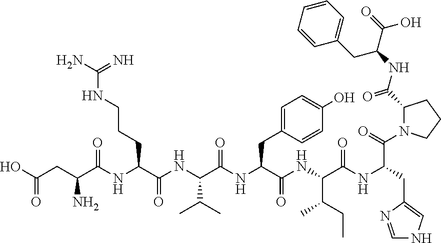 [SMILES: CC[C@H](C)[C@H](NC(=O)[C@H](Cc1ccc(O)cc1)NC(=O)[C@@H](NC(=O)[C@H](CCCNC(=N)N)NC(=O)[C@@H](N)CC(=O)O)C(C)C)C(=O)N[C@@H](Cc1c[nH]cn1)C(=O)N1CCC[C@H]1C(=O)N[C@@H](Cc1ccccc1)C(=O)O]

The molecular formula of angiotensin II shown above is C₅₀H₇₁N₁₃O₁₂ and the molecular weight is about 1045.5 Daltons.

In certain exemplary embodiments and without limitation, angiotensin II may be present in the form of a pharmaceutically acceptable salt; for example and without limitation, acetate, trifluoroacetate, ascorbate, aspartate, benzene sulfonate, nitrate, thiocyanate, hydrochloride, hydrobromide, sulphate, phosphate, acid phosphate, maleate, fumarate, lactate, tartrate, citrate, or gluconate salts. Other examples of pharmaceutically acceptable salts that may be used are described in the Handbook of Pharmaceutical Salts Properties, Selection, and Use; Wiley-VHC; 2nd rev. edition (2011), incorporated by reference in its entirety.

In various embodiments, angiotensin II may be 5-valine angiotensin II acetate, 5-valine angiotensin II amide, 5-L-isoleucine angiotensin II acetate, and 5-L-isoleucine angiotensin II amide, or a pharmaceutically acceptable salt thereof.

In some embodiments, the solvent in the formulations of angiotensin II is water, i.e., the solution of angiotensin II is an aqueous solution. The solution may be a saline solution, e.g., having an isotonic concentration of dissolved sodium chloride.

In particular embodiments, formulations of angiotensin II are lyophilized, which when reconstituted in an appropriate solvent (e.g., saline), produce therapeutic formulations suitable for administration into a subject. When the formulations of the invention are lyophilized, the concentrations of different ingredients discussed herein correspond to the concentrations in the therapeutic formulations produced after reconstituting the lyophilized formulations. In other embodiments, angiotensin II is provided in a vial as a lyophilized powder or cake, which when reconstituted in an appropriate solvent (e.g., saline for injection or infusion) produces therapeutic formulations that can be readily and rapidly used for the treatment of hypotension, e.g., by parenteral (e.g., intravenous) administration.

The amount of lyophilized angiotensin II in a vial may range from 0.5 mg to 100 mg per vial, but preferably would be in the range of 1 to 20 mg per vial and most preferably in the range of 2-10 mg per vial. In some embodiments, the vial includes 1 mg angiotensin II. In some embodiments, the vial includes 2 mg angiotensin II. In some embodiments, the vial includes 3 mg angiotensin II. In some embodiments, the vial includes 4 mg angiotensin II. In some embodiments, the vial includes 5 mg angiotensin II. In certain embodiments, the vial includes no less than 0.05 mg angiotensin II. In certain embodiments, the vial includes no less than 0.10 mg angiotensin II. In certain embodiments, the vial includes no less than 0.5 mg angiotensin II. In certain embodiments, the vial includes no less than 1.0 mg angiotensin II.

Angiotensin II, in the formulations described herein, can be at a concentration of about 1 to about 10 mg/mL, particularly, about: 1 mg/mL, 2 mg/mL, 3 mg/mL, 4 mg/mL, 5 mg/mL, 6 mg/mL, 7 mg/mL, 8 mg/mL, 9 mg/mL, or 10 mg/mL, even more particularly about: 1.0 mg/mL, 1.5 mg/mL, 2.0 mg/mL, 2.5 mg/mL, 3.0 mg/mL, 3.5 mg/mL, 4.0 mg/mL, 4.5 mg/mL, or 5.0 mg/mL. Such formulations may be reconstituted by dilution with a suitable diluent, such as saline (e.g., 0.9% saline) for injection or infusion, to a concentration appropriate for infusion. In some embodiments, the concentration of angiotensin II is 1 mg/mL. In some embodiments, the concentration of angiotensin II is 2 mg/mL. In some embodiments, the concentration of angiotensin II is 3 mg/mL. In some embodiments, the concentration of angiotensin II is 4 mg/mL. In some embodiments, the concentration of angiotensin II is 5 mg/mL.

### Indications

Certain embodiments of the invention also provide methods of treating low blood pressure in a subject by administering to the subject, a therapeutically effective (i.e., a pharmaceutically effective) amount of the formulations of angiotensin II described herein. Low blood pressure in a subject may arise from diseases including, but not limited to, acute kidney injury, circulatory shock, high-output shock, or sepsis-induced hypotension (e.g., septic shock).

In certain embodiments, formulations of the invention are diluted, e.g., in fluids suitable for parenteral administration, more particularly, in fluids suitable for intravenous administration, prior to administration to a patient. In certain embodiments, formulations of the invention are diluted in saline.

### Routes of Administration

The formulations disclosed herein can be administered in a variety of ways. In some embodiments, the compositions of the invention are suitable for parenteral administration. These compositions may be administered, for example, intraperitoneally, intravenously, intrarenally, or intrathecally. In some embodiments, the compositions are injected intravenously. One of skill in the art would appreciate that a method of administering a therapeutically effective amount of the formulations described herein would depend on factors, such as the age, weight, and physical condition of the patient being treated, and the disease or condition being treated. A skilled artisan would be able to select a method of administration optimal for a patient on a case-by-case basis.

The composition may be administered topically, enterally, or parenterally. The formulations of the invention can be administered subcutaneously, intravenously, intramuscularly, intranasally, by inhalation, orally, sublingually, by buccal administration, topically, transdermally, or transmucosally. The formulations of the invention can be administered by injection.

### Dosage Forms, Kits, and Containers

As used herein, the term “kit” refers to any delivery system for delivering materials. For example and without limitation, kits include one or more enclosures (e.g., boxes) containing the relevant materials (e.g., angiotensin II) and/or supporting materials (e.g., instructions, tubing, valves, or needles).

The term “vial” is used in accordance with its plain ordinary meaning, and refers to a vessel (e.g., bottle) capable of containing a liquid. In some embodiments, the vial is a glass container. In some such embodiments, the vial is a plastic container. In certain embodiments, the vial is a hermetically sealed vial. In some such embodiments, the vial is a 1 ml vial. In some embodiments, the vial is a 2 ml vial. In some embodiments, the vial is a 2.5 ml vial. In some embodiments, the vial is a 3 ml vial. In some embodiments, the vial is a 4 ml vial. In some embodiments, the vial is a 5 ml vial.

The term “container” is used in accordance with its plain ordinary meaning, and refers to an object capable of containing a liquid (e.g., bottle, box, bag). A container may be made of any suitable material, such as metal, plastic, wood, or glass.

Solutions or suspensions suitable for parenteral administration or intravenous infusion can include the following components: a sterile diluent such as water for injection, saline solution, or other synthetic solvents. The pH can be adjusted with acids or bases, such as hydrochloric acid or sodium hydroxide. The parenteral preparation can be enclosed in ampoules, disposable syringes or multiple dose vials made of glass or plastic. Formulations suitable for intravenous infusion may be formulated in accordance with principles and protocols for intravenous fluid therapy (e.g., see for example National Clinical Guideline Centre (UK). Intravenous Fluid Therapy: Intravenous Fluid Therapy in

Adults in Hospital [Internet]. London: Royal College of Physicians (UK); 2013 December (NICE Clinical Guidelines, No. 174.) 5, Available on the internet from the National Center for Biotechnology Information (NCBI) Bookshelf (Bookshelf ID: NBK333103), which is incorporated herein in its entirety for all purposes. For example, a formulation suitable for intravenous infusion includes a formulation capable of being delivered directly into the vein of a subject that when administered at a pharmaceutically effective amount is non-lethal and non-toxic.

The term “sterile” when applied to a composition or a vial is used in accordance with its plain ordinary meaning, and denotes the composition or vial is essentially free from pathogenic content (e.g., bacterium or bacterial components).

The term “pyrogen” is used in accordance with its plain ordinary meaning, and refers to an agent (e.g., compound, nucleic acid, or organism) that is capable of raising the body temperature of the subject (e.g., human) when in contact with the subject's blood. In certain embodiments, pyrogens produce a fever in the subject (e.g., human) when the pyrogen is present in the subject's blood. In certain embodiments, the pyrogen is an endotoxin. In certain embodiments, the pyrogen is a lipopolysaccharide.

The term “saline solution” is used in accordance with its plain ordinary meaning, and refers to an isotonic solution of sodium chloride and distilled water. In some embodiments, the saline solution has a pH of about 5.0 to about 7.5. In some such embodiments, the saline solution has a pH of about 5.5. In various embodiments, the saline solution is 0.9% saline solution (i.e., contains 9 g NaCl/L water).

The terms “infusion bag” and “IV infusion bag” are used in accordance with their plain ordinary meaning, and refer to a medical grade plastic bag capable of holding from about 100 ml to 5000 ml of fluid. In some embodiments, the infusion bag holds about 100 ml to 1000 ml of fluid. In various embodiments, the infusion bag is made from polyvinyl chloride (PCV) or ethylene vinyl acetate (EVA). In certain embodiments, the infusion bag is light protected (e.g., the infusion bag is composed of a material which affords UV protection within 89-99% of the wavelength range 175 nm to 525 nm).

In some embodiments, the dosage forms disclosed herein also include kits, packages and multicontainer units containing pharmaceutical compositions as described herein and/or means for administering them to treat subjects (e.g., treating hypotension in a subject in need thereof). In certain embodiments, these kits include a container or formulation that contains angiotensin II, or pharmaceutically acceptable salts thereof.

In certain embodiments, a typical kit may include a container, preferably a vial, bottle, or bag holding the angiotensin II or a pharmaceutically acceptable salt thereof, or a formulation thereof as disclosed herein, and (optionally) instructions, such as a product insert or label, directing the user to prepare an aqueous formulation suitable for administration to the subject. Preferably, the pharmaceutical formulation is for treating low blood pressure. In certain preferred embodiments, the container additionally includes sodium chloride and optionally mannitol, in aqueous solution at a pH of from or about 5.0 to 6.0, preferably at a pH of about 5.5. In certain such embodiments, the container is associated with instructions to dissolve and/or dilute the contents of the container(s). In some embodiments, the vial includes about 2.5 mg/mL (mg) angiotensin II in saline (e.g., 0.9% saline) and 25 mg/mL mannitol. In certain embodiments, the container includes mannitol. In alternative embodiments, the container does not include mannitol.

In yet further preferred embodiments, the invention provides a kit including a vial, bottle, infusion bag, or syringe containing a dosage form. The kit can further include instructions and accessories useful for diluting and/or administering the dosage form.

In certain embodiments, the kit comprises a number of separate containers (e.g., 2, 3, 4, 5, 6, 7, 8, 9, or 10 containers) of angiotensin II that, when combined, provide sufficient angiotensin II to treat a human suffering from low blood pressure over a course of treatment. The containers may be associated with instructions for preparing and/or administering the angiotensin II as disclosed herein. In preferred embodiments, the course of treatment lasts at least 6 to 48 hours. In particularly preferred embodiments, the course of treatment lasts at least 24 to 48 hours, and most preferably for at least 48 hours.

In preferred embodiments, the administration of angiotensin II, or pharmaceutically acceptable salts thereof, is administered continuously by uninterrupted infusion for at least 6, 12, 24, 48 hours, or more. Preferably, continuous, uninterrupted infusion is for 24 hours. Most preferably, continuous, uninterrupted infusion is for 48 hours.

Suitable containers for the disclosed dosage forms include glass vials, for example, nominal 50-125 ml vials, such as 100 ml vials, infusion bags, or syringes adapted for intravenous administration of said solution, each comprising suitable closure means. A container may be further enclosed or packaged in an opaque covering. The glass or polymer of which the container is formed can be colored, e.g., amber colored, to provide further shielding from light exposure. Accordingly, various embodiments of the invention provide a kit comprising a vial, infusion bag, or syringe, such as are described above, containing a dosage form, or a dosage form to be prepared by a method as described herein. The kit can further include instructions for preparing a formulation suitable for administration to a patient. In certain preferred embodiments, where the angiotensin II is provided as a lyophilisate or a concentrated solution (for dilution prior to administration), the vial is a 1 mL vial or a 2 mL vial.

Other embodiments provide a kit adapted for a single course of treatment comprising one, two, or more containers as described above enclosed in packaging material, for example polystyrene foam packaging adapted to protect the bottles from impact, light, extremes of temperature, and so forth. The kit can further include accessories useful for administration of the container contents such as tubing, valves, needles for IV administration, etc. A kit can further include instructional materials, such as instructions directing the dose or rate of administration. For example, a kit can comprise sufficient doses for a prolonged period, such as a day or a plurality of days, or can comprise multiple unit dosage forms for a single administration when the dose is to be administered at a lower rate or for a shorter duration. The multiple unit dosage forms can be packaged separately, but in proximity, as in a blister pack. In other embodiments, provided herein are a plurality of kits in a packaging adapted for shipping, for example, two courses of three containers each.

In some embodiments, the kit can also contain one or more containers of solution of a diluent or solvent, such as sterile water for injection, sterile saline, sterile buffer solutions and the like. The first container (angiotensin II) and second container can be provided with fluid delivery means to permit the administration to a patient of solutions prepared from the containers.

In some embodiments, contents of provided formulation in a container can be reconstituted or dissolved in a solvent (e.g., water or saline) to form a dose concentrate.

In other embodiments, kits may optionally further include a second container comprising appropriate solvent or diluent, and/or instructions for use of appropriate solvent or diluent for preparation of reconstituted formulation. In some embodiments, contents of provided formulation in a first container and solvent in a second container combine to form a unit dosage (e.g., a bolus) sufficient for a course of treatment. In some embodiments, contents of provided formulation in a first container and solvent in a second container combine to form a dose concentrate.

In still other embodiments, a third container comprising a suitable aqueous carrier for further dilution of a reconstitute or solution for preparation of administration to a subject via intravenous (IV) administration.

In some embodiments, a single container may comprise one or more compartments for containing a lyophilized formulation, appropriate liquid carrier for reconstitution, and/or appropriate aqueous carrier for dilution.

In some embodiments, a pharmaceutical kit comprises a lyophilized formulation in a reconstitution package or container wherein a needle-less exchange mechanism allows for combination of lyophilate and aqueous carrier for dilution and/or with isotonic diluent for preparation for intravenous administration. For example, in certain non-limiting examples, a lyophilized formulation of the invention may be utilized in conjunction with a MINIBAG® Plus reconstitution package system (Baxter), or an ADD VANTAGE® reconstitution package (Hospira) system.

In preferred embodiments, the kit includes (i) a unit dosage form containing a lyophilized powder including angiotensin II, or pharmaceutically acceptable salt thereof, and (ii) instructions for reconstituting the lyophilized powder with an aqueous solution to form a pharmaceutical composition suitable for administration to a subject.

In other embodiments, the kit includes (i) a unit dosage form containing a liquid concentrate including angiotensin II, or pharmaceutically acceptable salt thereof, and (ii) instructions for diluting the liquid concentrate with an aqueous solution to form a pharmaceutical composition suitable for injection into a subject. In various embodiments, the kit, container, or vial does not contain an additional vasopressor (e.g., no additional vasopressors beyond angiotensin II). In some embodiments, the kit, container, or vial does not contain methylene blue.

In preferred embodiments, the kit is adapted for providing a single course of treatment by combining one or more of the dosage forms further contained in packaging material. For example and without limitation, the kit can include three dosage form units, each dosage form unit providing 20 mL of a solution comprising 100 mg of angiotensin II, for a total of 300 mg angiotensin per kit, which suffices for at least one administration of a dose of angiotensin II of up to 300 mg. The packaging material of the kit can be light-protective in order to avoid photolytic decomposition of the angiotensin II. The kit can include packaging material such as shaped polystyrene foam that serves to protect the containers from damage, light, and thermal extremes. The kit can further include instruction means and labeling means, as well as accessories for administration of the container contents such as tubing, valves, or needles for IV administration.

In additional embodiments the dosage form can further be packaged in multiple dosage forms adapted to practice the methods of the invention. For example, two or three single-unit dosage forms can be packaged together as a “six-pack,” for example for shipment from a supplier to a medical facility providing treatment to patients, in a single container.

In further embodiments, the kit can include separately packaged and labeled multiple or single use containers of angiotensin II intended to be administered parenterally or by IV infusion.

In some embodiments, the contents of the container (e.g., vial) are substantially free of pyrogens (e.g., greater than 99% free of pyrogens).

### Methods

In an aspect is provided a method for treating hypotension in a subject, the method including administering to the subject a formulation as described herein, the contents of a vial as described herein, or the contents of a container as described herein.

In another aspect is provided a method for increasing blood pressure (e.g., the mean arterial pressure) in a subject the method including administering to the subject a formulation as described herein, the contents of a vial as described herein, or the contents of a container as described herein (e.g., wherein the increasing is relative to the blood pressure prior to administering the formulation, contents of the vial, or the contents of the container). In certain embodiments, the blood pressure is increased at least 10 to 15 mm Hg. In some embodiments, the blood pressure is increased 10 mm Hg to 75 mm Hg.

In yet another aspect is provided a method for maintaining the blood pressure (e.g., the mean arterial pressure) in a subject, the method including administering to the subject a formulation as described herein, the contents of a vial as described herein, or the contents of a container as described herein. In various embodiments, the blood pressure is maintained within 80 mm Hg and 110 mm Hg over a period of time (e.g., at least 8 hours). In certain embodiments, the blood pressure is maintained at a target range (e.g., within 80 mm Hg and 110 mm Hg) for about 8 to 24 hours.

### EXEMPLIFICATION

### Example 1: Use of Liquid Formulation to Facilitate the Treatment of Hypotension

Vials containing 2 milliliters of 2.5 mg/mL (mg) angiotensin II in 0.9% saline and 25 mg/mL mannitol were evaluated in a clinical study of angiotensin II in treating hypotension in distributive shock (Khanna et al., N Engl J Med 2017; 377:419-430). Patients were currently on other vasopressors but still hypotensive (mean arterial pressure (MAP)>65 mmHg). Administration of angiotensin II was begun at a rate of 20 ng/kg/min, and titrated up or down to achieve and maintain an increased MAP of 10 mmHg above baseline or at least 75 mmHg. For the first 3 hours of treatment, the other vasopressors were held constant and the maximum dose allowed of angiotensin II was 200 ng/kg/min. At 3 hours and 15 minutes, all vasopressors could be titrated to maintain a MAP between 65-75 mmHg and an angiotensin II rate of 1.25-40 ng/kg/min. Angiotensin II could be administered for up to 7 days in this study. In this study, about 1 vial of drug (5 mg) was sufficient to treat each patient for a 24-hour period, and patients were on the treatment for an average of 2 days. Most patients required at least 1 mg of drug per day.

A total of 344 patients were assigned to one of the two regimens; 321 received a study intervention (163 received angiotensin II, and 158 received placebo) and were included in the analysis. The primary end point (response with respect to mean arterial pressure at hour 3 after the start of infusion, with response defined as an increase from baseline of at least 10 mm Hg or an increase to at least 75 mm Hg, without an increase in the dose of background vasopressors) was reached by more patients in the angiotensin II group (114 of 163 patients, 69.9%) than in the placebo group (37 of 158 patients, 23.4%) (odds ratio, 7.95; 95% confidence interval [CI], 4.76 to 13.3; P<0.001).

The following are some specific numbered embodiments of the invention disclosed herein. These embodiments are exemplary and for the purpose of illustration only. It will be understood that the invention is not limited to the embodiments, but embraces all such forms and combinations thereof as come within the scope of the above disclosure.

Embodiment 1. A vial comprising angiotensin II or a pharmaceutically acceptable salt thereof, wherein the vial comprises about 0.5 to about 20 mg of angiotensin II.
Embodiment 2. The vial of embodiment 1, wherein the vial comprises about 1 to about 10 mg of angiotensin II.
Embodiment 3. The vial of embodiment 1, wherein the vial comprises about 2 to about 5 mg of angiotensin II.
Embodiment 4. The vial of any preceding embodiment, wherein the contents of the vial are sterile.
Embodiment 5. The vial of embodiment 4, wherein the contents of the vial are substantially free of pyrogens.
Embodiment 6. The vial of any preceding embodiment, wherein the angiotensin II or pharmaceutically acceptable salt thereof is provided as a lyophilisate.
Embodiment 7. A vial comprising a sterile, aqueous formulation of angiotensin II, or a pharmaceutically acceptable salt thereof, wherein the vial comprises about 0.5 to about 20 mg of angiotensin II.
Embodiment 8. The vial of embodiment 7, wherein the vial comprises about 1 to about 10 mg of angiotensin II.
Embodiment 9. The vial of embodiment 7, wherein the vial comprises about 2 to about 5 mg of angiotensin II.
Embodiment 10. The vial of any one of embodiments 7-9, wherein the formulation has a concentration of about 0.5 to about 20 mg/mL angiotensin II.
Embodiment 11. The vial of embodiment 10, wherein the formulation has a concentration of about 2 to about 10 mg/mL of angiotensin II.
Embodiment 12. The vial of embodiment 10, wherein the formulation has a concentration of about 2.5 mg/mL of angiotensin II.
Embodiment 13. The vial of any preceding embodiment, wherein the vial has a volume between 0.5 and 100 mL.
Embodiment 14. The vial of embodiment 13, wherein the vial has a volume of about 2 mL or about 1 mL.
Embodiment 15. A method for preparing a sterile, aqueous formulation of angiotensin II, or a pharmaceutically acceptable salt thereof, suitable for intravenous infusion, comprising dissolving the contents of the vial of any preceding embodiment in a solvent.
Embodiment 16. The method of embodiment 15, wherein the formulation comprises angiotensin II, or a pharmaceutically acceptable salt thereof, at a concentration of about 0.00005 mg/mL to about 0.01 mg/mL.
Embodiment 17. The method of embodiment 15 or 16, wherein the solvent is a saline solution.
Embodiment 18. A sterile, aqueous formulation of angiotensin II made by the method of any one of embodiments 15-17.
Embodiment 19. An infusion bag comprising the formulation of embodiment 18.
Embodiment 20. A kit comprising (i) a vial of any one of embodiments 1-14, and (ii) instructions for diluting the contents of the vial with a solvent to achieve an aqueous formulation suitable for IV infusion into a subject.
Embodiment 21. The kit of embodiment 20, comprising 2 to 5 vials of any one of embodiments 1-14, and the instructions are for combining the contents of at least 2 vials to achieve the formulation.
Embodiment 22. A container comprising a sterile, aqueous formulation of angiotensin II, or a pharmaceutically acceptable salt thereof, suitable for intravenous infusion, wherein the formulation comprises the angiotensin II, or a pharmaceutically acceptable salt thereof, at a concentration of about 0.00005 mg/mL to about 0.01 mg/mL, and wherein the container comprises about 0.5 to about 20 mg of angiotensin II.
Embodiment 23. The container of embodiment 22, wherein the container comprises about 1 to about 10 mg of angiotensin II.
Embodiment 24. The container of embodiment 22, wherein the container comprises about 2 to about 5 mg of angiotensin II.
Embodiment 25. The container of any one of embodiments 22-24, wherein the container is an infusion bag.
Embodiment 26. A method for treating hypotension in a subject, comprising administering to the subject a formulation of embodiment 18, the contents of a vial of any one of embodiments 1-14, or the contents of a container of any one of embodiments 22-25.
Embodiment 27. A method for increasing blood pressure in a subject, comprising administering to the subject a formulation of embodiment 18, the contents of a vial of any one of embodiments 1-14, or the contents of a container of any one of embodiments 22-25.
Embodiment 28. A method for maintaining a desired blood pressure in a subject, comprising administering to the subject a formulation of embodiment 18, the contents of a vial of any one of embodiments 1-14, or the contents of a container of any one of embodiments 22-25.
Embodiment 29. The method of any one of embodiments 26-28, wherein the method comprises diluting the contents of a vial of any one of embodiments 1-14 in a pharmaceutically acceptable excipient and administering the diluted formulation to the subject.
Embodiment 30. The method of any one of embodiments 26-29, comprising administering the formulation intravenously.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

1-30. (canceled)

31. A dosage form for formulating an intravenous infusion treatment of distributive shock in a human patient comprising:
an amount of human angiotensin II or a pharmaceutically acceptable salt thereof (5-L-isoleucine angiotensin II) disposed within a hermetically sealed vial for formulating an intravenous infusion treatment of the patient;
wherein the amount is about 0.5 to about 20 mg of human angiotensin II in the dosage form, and wherein the dosage form is sterile.

32. The dosage form of claim 31, comprising about 0.5 to about 10 mg of human angiotensin II or a pharmaceutically acceptable salt thereof.

33. The dosage form of claim 31, comprising about 0.5 to about 5 mg of human angiotensin II or a pharmaceutically acceptable salt thereof.

34. The dosage form of claim 31, comprising about 0.5 to about 2.5 mg of human angiotensin II or a pharmaceutically acceptable salt thereof.

35. The dosage form of claim 31, wherein the human angiotensin II or pharmaceutically acceptable salt thereof is provided as a lyophilisate.

36. The dosage form of claim 35, wherein a needle-less exchange mechanism allows for combination of the lyophisate in the hermetically sealed vial and an aqueous carrier for dilution and/or with isotonic diluent for preparation for the intravenous administration.

37. The dosage form of claim 31, wherein the human angiotensin II or pharmaceutically acceptable salt thereof is provided as an acetate salt.

38. The dosage form of claim 31, wherein the amount of human angiotensin II or pharmaceutically acceptable salt thereof is in an aqueous solution at a concentration of about 0.5 to about 10 mg/mL.

39. The dosage form of claim 38, wherein the concentration of human angiotensin II or pharmaceutically acceptable salt thereof is about 0.5 to about 2.5 mg/mL.

40. The dosage form of claim 31, wherein the hermetically sealed vial has a volume between 0.5 and 100 mL.

41. The dosage form of claim 40, wherein the hermetically sealed vial has a volume between 1 and 20 mL.

42. The dosage form of claim 40, wherein the hermetically sealed vial has a volume of 1 mL.

43. The dosage form of claim 31, wherein said dosage form is substantially free of pyrogens.

44. The dosage form of claim 31, wherein the dosage form is for a single use.

45. The dosage form of claim 31, wherein the dosage form is for a single dose.

46. The dosage form of claim 31, wherein the dosage form is for administration to a single subject.

47. A dosage form for formulating an intravenous infusion treatment of distributive shock in a single human patient comprising:
comprising an amount of human angiotensin II or a pharmaceutically acceptable salt thereof (5-L-isoleucine angiotensin II) disposed within a hermetically sealed vial for formulating an intravenous infusion treatment of the patient;
wherein the amount is about 0.5 to about 20 mg of human angiotensin II in the dosage form, and wherein the dosage form is sterile.

48. The dosage form of claim 47, comprising about 0.5 to about 10 mg of human angiotensin II or a pharmaceutically acceptable salt thereof.

49. The dosage form of claim 47, comprising about 0.5 to about 5 mg of human angiotensin II or a pharmaceutically acceptable salt thereof.

50. The dosage form of claim 47, comprising about 0.5 to about 2.5 mg of human angiotensin II or a pharmaceutically acceptable salt thereof.

51. The dosage form of claim 47, wherein the human angiotensin II or pharmaceutically acceptable salt thereof is provided as a lyophilisate.

52. The dosage form of claim 47, wherein a needle-less exchange mechanism allows for combination of the lyophilisate in the hermetically sealed vial and an aqueous carrier for dilution and/or with isotonic diluent for preparation for the intravenous administration.

53. The dosage form of claim 47, wherein the human angiotensin II or pharmaceutically acceptable salt thereof is provided as an acetate salt.

54. The dosage form of claim 47, wherein the amount of human angiotensin II or pharmaceutically acceptable salt thereof is in an aqueous solution at a concentration of about 0.5 to about 10 mg/mL.

55. The dosage form of claim 54, wherein the concentration of human angiotensin II or pharmaceutically acceptable salt thereof is about 0.5 to about 2.5 mg/mL.

56. The dosage form of claim 47, wherein the hermetically sealed vial has a volume between 0.5 and 100 mL.

57. The dosage form of claim 56, wherein the container has a volume between 1 and 20 mL.

58. The dosage form of claim 56, wherein the container has a volume of 1 mL.

59. The dosage form of claim 47, wherein said dosage form is substantially free of pyrogens.

60. The dosage form of claim 47, wherein the intravenous infusion treatment is administered as a bolus or as continuous infusion.

61. The dosage form of claim 59, wherein the continuous infusion is for at least 6 hours, at least 12 hours, at least 24 hours, or at least 48 hours.
